(19) United States
(12) Patent Application Publication
(10) Pub. No.: US 2022/0357279 A1
(43) Pub. Date: Nov. 10, 2022

(54) DUAL WAVELENGTH COMBINED FINGERPRINT AND HIGH WAVENUMBER RAMAN SPECTROSCOPY AND APPLICATIONS OF SAME

(71) Applicant: VANDERBILT UNIVERSITY, Nashville, TN (US)

(72) Inventors: Laura E. Masson, Nashville, TN (US); Anita Mahadevan-Jansen, Nashville, TN (US)

(21) Appl. No.: 17/691,567

(22) Filed: Mar. 10, 2022

Related U.S. Application Data
(60) Provisional application No. 63/158,962, filed on Mar. 10, 2021.

(51) Int. Cl. G01N 21/65; G01J 3/44; A61B 5/00; A61B 5/1455; A61B 5/256; G01J 3/10; G01J 3/02; G01J 3/28

(52) U.S. Cl. CPC G01N 21/65; G01J 3/4412; A61B 5/0075; A61B 5/1455; A61B 5/256; G01J 3/10; G01J 3/0218; G01J 3/021; G01J 3/2803; G01J 2003/2859

(57) ABSTRACT

A system for real-time assessment of systemic hydration includes a light source configured to operably emit light of first and second wavelengths; means for delivering the emitted light to a target site to excite at least one first spot at the target site, and collecting Raman scattering light scattered from the target site at a plurality of second spots; a detector coupled with said means for obtaining a plurality of spatially offset Raman spectra from the collected Raman scattering light, each spatially offset Raman spectrum corresponding to a respective second spot of the target site and associated with a depth of tissues at which the Raman scattering light is scattered; and a controller configured to process the plurality of spatially offset Raman spectra so as to identify spectral features from the plurality of spatially offset Raman spectra, and assess systemic hydration from the identified spectral features.

### CROSS-REFERENCE TO RELATED PATENT APPLICATION

This application claims priority to and the benefit of U.S. Provisional Patent Application Ser. No. 63/158,962, filed Mar. 10, 2021, which is incorporated herein by reference in its entirety.

### STATEMENT AS TO RIGHTS UNDER FEDERALLY-SPONSORED RESEARCH

This invention is made with government support under Grant No. W81XWH-20-2-0064 awarded by the Department of Defense (DOD) Army Combat Readiness Research Program. The government has certain rights in the invention.

### FIELD OF THE INVENTION

The invention relates generally to Raman spectroscopy in biomedical applications, and more particularly, to dual wavelength combined fingerprint and high wavenumber Raman spectroscopy and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

For people who are subject to intense training regimens or exposed to extreme conditions, accurate physiological monitoring is essential to ensure that peak performance is maintained. This is particularly true for military personnel—from training to continued deployment. The development of wearable sensors with multiple capabilities, including physiological monitoring of parameters such as heart rate, blood pressure, respiration, and hydration, has been identified as an area of research priority by the Combat Readiness-Medical Research Program. However, most current sensors suffer from poor accuracy and do not typically include hydration.

Dehydration has a widespread physical impact on the body in addition to affecting cognitive function, and by the time a person begins to experience thirst, it is too late to prevent the resulting effects. Dehydration is a challenge that lacks accurate, non-invasive sensors and is critical in various populations including the elderly, athletes and the military. The military population is at especially high risk for dehydration since they work in extreme environments, lack access to water on long missions, and have to sustain high levels of physical activity. A body water deficit, also termed hypohydration, has been shown to impact cognitive function when water loss exceeds 2% of total body weight. Signs and symptoms of dehydration in adults include extreme thirst, fatigue, dizziness and confusion. Over time, dehydration can lead to complications such as heat injury (i.e., heat exhaustion or heat stroke), urinary and kidney problems, seizures, and hypovolemic shock caused by low blood volume. Consequently, maintaining proper hydration, and being able to monitor this in real time, is critical to the success of military training and missions.

In practice, medical professionals can often diagnose dehydration on the basis of physical signs and symptoms presented. It should be emphasized that by the time symptoms are diagnosable, the dehydration in an individual is likely significantly advanced and the patient is experiencing low blood pressure, a faster than normal heart rate, and reduced blood flow to extremities. To confirm this diagnosis and pinpoint the degree of dehydration, professionals may use blood tests or urinalysis. Again, once these symptoms are determined, all that can be done is to replace lost fluids and lost electrolytes. However, negative impact to soldiers, sailors, or airmen during training or field operations has already occurred. The most accurate methods for assessing systemic hydration levels rely on blood testing, which is invasive and both time and resource intensive. The change in plasma volume is often used as a surrogate measure of total body water and is calculated based on the change in hemoglobin concentration and the change in hematocrit concentration in a blood sample compared to those same values based on a “baseline” blood sample. This requires availability of a baseline and access to this information at the time of the dehydration event. The level of plasma osmolality and sodium concentration can also be calculated using a blood sample, where both plasma sodium and osmolality are significantly elevated in cases of dehydration. Though reliable, these methods require a blood draw, which is not feasible for continuous monitoring, particularly in the field. Another highly accurate approach for determining total body water content is stable isotope dilution, in which a person consumes water labeled with a stable isotope such as deuterium or tritium, and its dilution with the body's inter- and extra-cellular water content is determined based on analysis of a saliva, urine, or blood sample several hours after consumption. The large timeframe required for completing such measurements is prohibitive to military application. Neutron activation analysis has been used to determine the concentration of chloride, potassium, and sodium, which can in turn be used to calculate the extracellular fluid volume, intracellular fluid volume, and total body water, although this approach requires highly specialized equipment and technical expertise. The concentration of arginine vasopressin, a hormone which is secreted in response to dehydration, can be measured in the plasma, but requires a 3-day laboratory test with specialized equipment. Change in body weight can also be used as an indirect measure of hydration, though numerous other variables can impact this measurement and thus decrease its accuracy.

Various urine-based metrics for hydration assessment have been developed with a wide range of accuracies. The simplest of these is based on colorimetric analysis of urine, and while effective at assessing moderate hypo- or hyperhydration, it fails to provide an accurate metric for patients who are critically ill or severely dehydrated. More quantitative metrics for evaluating hydration based on urinalysis include urine specific gravity and urine osmolality, each of which provide a measure of solute concentration in urine. Though less invasive than blood testing, urinalysis requires time, and in many cases, laboratory equipment, to complete, in addition to providing periodic measurements rather than continuous monitoring. Bioelectrical impedance analysis is a noninvasive method for estimating total body water which measures the body's resistance to low levels of electrical current at various frequencies. The National Institute of Health stated that bioelectrical impedance provides a reliable estimate of hydration status under most conditions, although such measures can also be impacted by numerous factors such as skin temperature, food or drink consumption, and body posture before the measurement, and therefore requires standardization steps. This form of hydration analysis can be implemented in a field setting using a portable bioelectrical impedance device, and wearable devices which make use of this technique are currently in development. Many of these methods suffer from poor accuracy, are difficult to implement in field or combat settings, and do not provide continuous monitoring.

Wearable technologies for hydration monitoring have sought to fill these gaps left by existing technologies. In this active area of research, scientists and engineers have employed various technological approaches including visual/optical, electromagnetic, chemical, and acoustical measurements to sense hydration. One wearable sensor for both glucose and hydration has been developed using an “optical cardiogram” in which green light is pulsed onto the skin and a sensor detects the resulting speckle pattern, then determines the temporal correlation between successive speckle patterns. However, validation studies of this technique have shown inconsistent results and therefore the accuracy of this approach is unclear. Several studies have sought to apply near-infrared spectroscopy (NIRS) to evaluate skin moisture content, although correlations between these measures and total body water have not been established. Wearable configurations of the bioelectric impedance approach have also been developed, for example, using multiple pairs of electrodes to measure impedance of the skin at various depths. Several wristbands with hydration monitoring capabilities have recently become commercially available or will soon be brought to market. These employ visual/optical, electromagnetic, and chemical sensing modalities with various degrees of success. For instance, the commercially available Healbe GoBe2 wristband claims to estimate the body's water balance based on input from a bioimpedance sensor paired with an accelerometer and factors such as gender, weight, and age. In addition to being plagued by the previously mentioned challenges associated with bioimpedance measurements, the accuracy may be affected by the presence of sweat or moisture on the hydration sensor, and rather than outputting a quantitative reading of hydration level, the device merely reports whether hydration is normal, low, or not available. Other wristband-based technologies also employ an impedance approach, including one using conformal nanowire electrodes, although this approach is demonstrated to measure skin moisture rather than systemic hydration. Sweat-based hydration sensing has also been under development in wearable formats such as wristbands and patches. Companies such as General Electric and Gatorade as well as academic institutions are developing sweat sensing patches which employ a microfluidic approach to measure sweat parameters such as electrolyte (sodium and potassium) concentration, glucose concentration, and sweat rate. The main limitation of sweat sensing is that it provides an indirect measure of total body water and as such is unable to detect real-time hydration variability. Further, it is likely that change in electrolyte concentration is a secondary effect and delayed relative to the primary dehydration event, therefore unreliable in extreme conditions. However, the patch format of such devices facilitates easy implementation and nearly continuous monitoring during physical activity, which may be ideally suited for athletics and other applications. However, for military personnel participating in high-stakes operations, real-time monitoring of even minute changes in hydration is necessary, and sweat analysis is unable to provide this. Further, the inability of this technique to evaluate hydration status in cases where a person is not sweating prohibits the acquisition of a baseline hydration measurement and thus limits the accuracy of the approach.

In light of gaps in current technology, there is a need for a hydration monitoring technology for individuals that can provide accurate real-time results and relies on direct system hydration. The ideal solution will be wearable, durable, easy-to-use, field-ready, and able to transmit results to remote location in a secure manner.

### SUMMARY OF THE INVENTION

In view of aforementioned deficiencies and inadequacies, one of the objectives of this invention is to develop a robust, non-invasive, wearable, and easy to use device that measures in vivo tissue Raman spectra with an accompanying model for calculating water content to enable accurate and real-time systemic hydration monitoring in high performance and endurance individuals, The device utilizes multiple spectral features, including the five component peaks of the high wavenumber water band and peaks in the fingerprint region, in order to accurately quantify total body water.

In one aspect, the invention relates to a system for real-time assessment of systemic hydration. The system includes a light source configured to operably emit light of first and second wavelengths; means for delivering the emitted light to a target site to excite at least one first spot at the target site, and collecting Raman scattering light scattered from the target site at a plurality of second spots in response to excitation by the light; a detector coupled with said means for obtaining a plurality of Raman spectra from the collected Raman scattering light, wherein each Raman spectrum is corresponding to a respective second spot of the target site, and associated with a depth of tissues at which the Raman scattering light is scattered; and a controller coupled with the detector and configured to process the plurality of Raman spectra so as to identify spectral features from the plurality of Raman spectra, and assess systemic hydration from the identified spectral features.

In one embodiment, the light source comprise a dual wavelength laser module, wherein the first and second wavelengths are adapted such that when excited by the first wavelength light, the Raman scattering light corresponds to a fingerprint region; and when excited by the second wavelength light, the Raman scattering light corresponds to a high wavenumber region.

In one embodiment, the first wavelength is about 785 nm, and the second wavelength is in a range of about 660-700 nm.

In one embodiment, the dual wavelength laser module is configured to be operably switchable between the first wavelength and the second wavelength.

In one embodiment, the system is capable of sequential or simultaneous acquisition of fingerprint and high wavenumber spectra, and collecting signal from multiple depths within the target site in a single acquisition.

In one embodiment, each second spot is apart from the at least one first spot so as to define a source-detection (S-D) offset distance between the at least one first spot excited with the light and the second spot from which the Raman scattering light is collected.

In one embodiment, the at least one first spot comprises a plurality of first spots.

In one embodiment, said means comprises an optical probe having a working end, coupled with the light source and configured to deliver the light to the target site to excite the at least one first spot proximal to the working end, and collect from the working end Raman scattering light scattered from the target site at one or more of the plurality of second spots in response to excitation.

In one embodiment, the optical probe comprises at least one source channel configured to deliver the light to the target site to excite the at least one first spot proximal to the working end; and at least one collection channel configured to collect from the working end Raman scattering light scattered from the target site at the one or more of the plurality of second spots in response to excitation.

In one embodiment, the optical probe comprises at least one first fiber configured to deliver the light to the target site to excite the at least one first spot proximal to the working end; and a plurality of second fibers configured to collect from the working end Raman scattering light scattered from the target site.

In one embodiment, the at least one first fiber and the plurality of second fibers are spatially arranged in a row, a matrix, a wing, or a ring form.

In one embodiment, the plurality of second fibers is spatially arranged in one or more collection rings surrounding the at least one first fiber.

In one embodiment, the plurality of second fibers is spatially arranged in three collection rings originated from the at least one first fiber with radii R1, R2 and R3 respectively.

In one embodiment, the optical probe is configured such that as the distance from the center of the probe increases, the number of collection fibers also increases to compensate for decreasing signal intensity with increasing offset.

In one embodiment, the inner, middle and outer rings are configured to measure hydration parameters from different depths.

In one embodiment, the fiber optic probe further comprises a shortpass or bandpass filter coupled to the at least one first fiber for delivering either the first wavelength light or the second wavelength light to the target site while preventing extraneous wavelengths of light from being transmitted.

In one embodiment, the shortpass or bandpass filter has a bandwidth of about 600-800 nm.

In one embodiment, the fiber optic probe further comprises a blocking filter coupled to the plurality of second fibers for preventing backscattered excitation light from being collected.

In one embodiment, the blockingfilter has an cut-on wavelength of about 800 nm.

In one embodiment, the detector comprises a spectrograph and/or a sensing member.

In one embodiment, the sensing member comprises at least one charge-coupled device (CCD), at least one complementary metal oxide semiconductor (CMOS), and/or at least one photodiode.

In one embodiment, the spectral features are associated with water or electrolytes, or metabolic products, pus, or bacteria, or cells, and include spectral peaks in the high wavenumber region and the fingerprint region.

In one embodiment, the controller is configured to correlate the identified peaks and spectral ratios for each depth of collection corresponding to the inner, outer and middle rings of the optical probe, so as to determine the optimal depth of measurement for hydration assessment based on these correlation values.

In one embodiment, the controller is configured to analyze the changes associated with hydration level using multivariate statistical, machine learning, deep learning or artificial intelligence (AI) approaches.

In one embodiment, the analysis utilizes generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, Xis a matrix containing independent variables of BMI, age and temperature, p is a vector containing weight coefficients of the independent variables, and E is a residual error in the GLM, wherein a linear least squares regression is performed to choose p coefficients such that E is minimized.

In one embodiment, the controller is configured to perform Voigtian decomposition of the high wavenumber region of the Raman spectra into the spectral peaks including five component water peaks and an N-H peak in the same region, so as to analyze the distribution of fully-bound, partially bound, and free water molecules in the target site.

In one embodiment, the spectral features further includes spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

In another aspect, the invention relates to a method for real-time assessment of systemic hydration, comprising exciting a target site at at least one first spot with light of first and second wavelengths; collecting Raman scattering light from the target site at a plurality of second spots, respectively, in response to illumination by the light; obtaining the plurality of Raman spectra from the collected Raman scattering light, wherein each Raman spectrum is corresponding to a respective second spot of the target site, and associated with a depth of tissues at which the Raman light is scattered; identifying spectral features from the plurality of Raman spectra; and determining systemic hydration from the identified spectral features.

In one embodiment, the first and second wavelengths are adapted such that when excited by the first wavelength light, the Raman scattering light corresponds to a fingerprint region; and when excited by the second wavelength light, the Raman scattering light corresponds to a high wavenumber region.

In one embodiment, the first wavelength is about 785 nm, and the second wavelength is in a range of about 660-700 nm. In one embodiment, the exciting step comprises exciting the target site at the at least one first spot with the first wavelength light and the second wavelength light sequentially, so that the fingerprint and high wavenumber spectra are sequentially acquired, and signals from multiple depths within the target site are collected in a single acquisition.

In one embodiment, the at least one first spot comprises a plurality of first spots.

In one embodiment, the exciting and collecting steps are performed with an optical probe having a working end.

In one embodiment, the optical probe comprises: at least one source channel configured to deliver the light to the target site to excite the at least one first spot proximal to the working end; and at least one collection channel configured to collect from the working end Raman scattering light scattered from the target site at one or more of the plurality of second spots in response to excitation.

In one embodiment, the optical probe comprises: at least one first fiber configured to deliver the light to the target site to excite the at least one first spot proximal to the working end; and a plurality of second fibers configured to collect from the working end Raman scattering light scattered from the target site at the plurality of second spots in response to excitation.

In one embodiment, the at least one first fiber and the plurality of second fibers are spatially arranged in a row, a matrix, a wing, or a ring form.

In one embodiment, the plurality of second fibers is spatially arranged in one or more collection rings surrounding the at least one first fiber.

In one embodiment, the plurality of second fibers is spatially arranged in three collection rings originated from the at least one first fiber with radii R1, R2 and R3 respectively.

In one embodiment, the optical probe is configured such that as the distance from the center of the probe increases, the number of collection fibers also increases to compensate for decreasing signal intensity with increasing offset.

In one embodiment, the inner ring, middle and outer rings are configured to measure hydration parameters from different depths.

In one embodiment, the fiber optic probe further comprises a shortpass or bandpass filter coupled to the at least one first fiber for delivering either the first wavelength light or the second wavelength light to the target site while preventing extraneous wavelengths of light from being transmitted.

In one embodiment, the shortpass or bandpass filter has a bandwidth of about 600-800 nm.

In one embodiment, the fiber optic probe further comprises a blocking filter coupled to the plurality of second fibers for preventing backscattered excitation light from being collected.

In one embodiment, the blocking filter has an cut-on wavelength of about 800 nm. In one embodiment, the obtaining step is performed with a detector comprising a spectrograph and/or a sensing member.

In one embodiment, the sensing member comprises at least one charge-coupled device (CCD), at least one complementary metal oxide semiconductor (CMOS), and/or at least one at least one photodiode.

In one embodiment, the spectral features are associated with water, or electrolytes, or metabolic products, pus, or bacteria, or cells, and include spectral peaks in the high wavenumber region and the fingerprint region.

In one embodiment, the method further comprises correlating the identified peaks and spectral ratios for each depth of collection corresponding to the inner, outer and middle rings of the optical probe, so as to determine the optimal depth of measurement for hydration assessment based on these correlation values.

In one embodiment, the method further comprises analyzing the changes associated with hydration level using multivariate statistical, machine learning, deep learning or artificial intelligence (AI) approaches.

In one embodiment, the analyzing step is performed with generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, Xis a matrix containing independent variables of BMI, age and temperature, p is a vector containing weight coefficients of the independent variables, and E is a residual error in the GLM, wherein a linear least squares regression is performed to choose p coefficients such that E is minimized.

In one embodiment, the method further comprises performing Voigtian decomposition of the high wavenumber region of the Raman spectra into the spectral peaks including five component water peaks and an N-H peak in the same region, so as to analyze the distribution of fully-bound, partially bound, and free water molecules in the target site.

In one embodiment, the spectral features further includes spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

In yet another aspect, the invention relates to a device using Raman spectral features for real-time assessment of systemic hydration of a subject in biomedical applications. The Raman spectral features are associated with water, electrolytes and metabolic products and include spectral peaks in the high wavenumber region and the fingerprint region. The device in one embodiment comprises a light source configured to emit light to excite a target site in the subject with an excitation wavelength that is determined based on wavenumber locations of the spectral peaks to be detected; a filter configured, for each feature to be detected, to pass Raman scattering light of the wavenumber range at which that feature falls with respect to the given excitation wavelength, wherein the Raman scattering light is scattered from the target site in response to excitation by the light; a detector configured to detect signals of the Raman scattering light passing through the filter; and a controller configured to operate the light source and process the detected signals by the detector to determine systemic hydration of a subject in real time.

In one embodiment, the light source comprises a VCSEL-technology based single monochromatic light source.

In one embodiment, the device further comprises dichroic mirrors (DMs) configured to direct the path of the emitted light.

In one embodiment, the device further comprises an alert configured to generate an audible signal and/or a visible signal whenever the measured hydration level falls below what is determined to be normal for the subject.

In one embodiment, the detector comprises at least one CCD, at least one CMOS, and/or at least one photodiode.

In one embodiment, the filter comprises at least one linear variable filter (LVF), and/or at least one high-throughput narrow bandpass filter.

In one embodiment, the detector comprises a an avalanche photodiode (APD), and/or a cooled linear diode array.

In one embodiment, the device further comprises one or more slit apertures located between the filter and the detector.

In one embodiment, the device is configured to accurately and noninvasively measure systemic hydration in a user and to send the measured results to one or more external devices for real-time remote monitoring, and/or data collection and storage.

In one embodiment, the device is configured to be wearable and portable.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIGS. 1A-1B show schematically a combined fingerprint (FP) and high wavenumber (HW) spatially offset Raman spectroscopy (SORS) system according to embodiments of the invention.

FIG. 2A shows schematically a cross-section review of an optical probe of the SORS system according to embodiments of the invention.

FIG. 2B shows schematically the SORS according to embodiments of the invention.

FIG. 3 shows schematically a portable device according to embodiments of the invention.

FIGS. 4A-4D show preliminary experimental data according to embodiments of the invention. FIG. 4A: Fingerprint Raman spectra of gelatin-based phantoms of varying concentrations. FIG. 4B: High wavenumber Raman spectra of gelatin-based phantoms of varying concentrations, demonstrating an increase in the water band with decreasing concentration. FIG. 4C: Ratio of the area under the curve of the water peak (3035 to 3680 cm⁻¹) to the area under the curve of the entire high wavenumber spectrum (2850 to 3680 cm⁻¹). FIG. 4D: Example of Voigtian fitting to the high wavenumber water band of gelatin phantom.

FIG. 5 show peak ratio of area under the curve 2910-2985 cm⁻¹ to area under the curve 3350-3550 cm⁻¹ as a function of percent water in bottom layer and thickness of upper layer according to embodiments of the invention.

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “living subject” refers to a human being such as a patient, or a mammal animal such as a monkey.

As used in this disclosure, “charge-coupled device” or “CCD” refers to an analog shift register that enables the transportation of analog signals (electric charges) through successive stages (capacitors), controlled by a clock signal. Charge-coupled devices can be used as a form of memory or for delaying samples of analog signals. Today, they are most widely used in arrays of photoelectric light sensors to serialize parallel analog signals. In a CCD for capturing images, there is a photoactive region (an epitaxial layer of silicon), and a transmission region made out of a shift register (the CCD, properly speaking).

An image is projected through a lens onto the capacitor array (the photoactive region), causing each capacitor to accumulate an electric charge proportional to the light intensity at that location. A one-dimensional array, used in line-scan cameras, captures a single slice of the image, while a two-dimensional array, used in video and still cameras, captures a two-dimensional picture corresponding to the scene projected onto the focal plane of the sensor. Once the array has been exposed to the image, a control circuit causes each capacitor to transfer its contents to its neighbor (operating as a shift register). The last capacitor in the array dumps its charge into a charge amplifier, which converts the charge into a voltage. By repeating this process, the controlling circuit converts the entire semiconductor contents of the array to a sequence of voltages, which it samples, digitizes and stores in some form of memory.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Raman spectroscopy (RS) is an inelastic light scattering technique that probes the different vibrational modes of molecules and yields a sample-specific molecular fingerprint, from which biochemical composition can be inferred. The fingerprint Raman bands (500-1800 cm⁻¹) correspond to bonds in lipids, proteins, blood and nucleic acids, and the high wavenumber Raman bands (2500-3800 cm⁻¹) can be related to partially and fully bound water as well as lipids and proteins in the tissue. Due to its high sensitivity and specificity and noninvasive nature, RS has been increasingly used in biomedical applications. Fingerprint RS has been used to detect disease in various organs such as the bladder, esophagus, skin, breast, and gastrointestinal tract. High wavenumber RS has been used to study hydration in tissues such as bone and skin.

The sensitivity of the high wavenumber region to water molecules and related hydrogen bonding and its ability to probe water-tissue interactions in a quantitative manner while simultaneously providing information about a variety of other tissue components make this method ideally suited for accurate, noninvasive hydration monitoring. Numerous studies have demonstrated the ability of Raman spectral metrics based on the high wavenumber region to accurately quantify hydration in phantoms as well as tissues such as bone and skin. Beyond tissue water quantification, high wavenumber Raman spectroscopy is capable of discriminating between fully hydrogen bound, partially hydrogen bound, and unbound water molecules based on deconvolution of the water band into its five component peaks. The relative amplitudes of these peaks have been shown to shift in response to varying concentrations of electrolytes, which is of particular importance for monitoring systemic hydration levels in high performance and endurance individuals such as military personnel.

One of the objectives of this invention is to develop a robust, non-invasive, wearable, and easy to use device that measures in vivo tissue Raman spectra with an accompanying model for calculating water content to enable accurate and real-time systemic hydration monitoring in high performance and endurance individuals. The device in one embodiment utilizes multiple spectral features, including the five component peaks of the high wavenumber water band and peaks in the fingerprint region, to accurately quantify total body water.

Spatially offset Raman spectroscopy, known as SORS, is a form of conventional Raman spectroscopy in which the excitation source and detector are separated by some spatial offset. As the distance between the source and detector increases, on average the photons have travelled deeper into the tissue before undergoing scattering events and returning to the detector, thus enabling the collection of biochemical information from deeper layers of tissue, on the order of several centimeters. This approach has previously been employed to detect breast tumors and microcalicifications. The ability to probe subsurface tissue layers is critical for hydration sensing as the goal is not to measure superficial skin hydration alone, but to obtain spectra that are indicative of the total body water. By employing the SORS approach, we are able to optimize the probing depth to most accurately quantify total body water and therefore systemic hydration, and to account for the effects of superficial skin moisture.

In one aspect, the invention relates to a novel dual wavelength combined fingerprint and high wavenumber Raman system that utilizes two different excitation wavelengths for sequential fingerprint and high wavenumber Raman spectroscopy. Combining the SORS with the dual wavelength, dual region approach allows one to probe systemic hydration directly and thus quantify water content in tissues. The SORS enables subsurface measurements indicative of systemic hydration rather than superficial skin hydration, and the dual region approach allows us to simultaneously quantify multiple markers of hydration status, such as water and electrolyte content, for improved accuracy. The primary goals of this invention therefore include, but are not limited to: (1) characterizing hydration using combined fingerprint and high wavenumber SORS measurements in vivo, (2) developing a model which incorporates relevant spectral features in order to accurately quantify human systemic hydration levels, and (3) developing a wearable device for timely and routine data acquisition and determination of dehydration onset. By using Raman spectroscopy, the early onset of dehydration in humans can be detected prior to apparent changes evident in sweat analysis and would eliminate the need for blood analysis, both of which are difficult to implement in high stress conditions.

In certain embodiments, the novel dual wavelength combined fingerprint and high wavenumber Raman system is tested in individuals such as military personnel during training at various states of hydration. Spectra are analyzed alongside the gold standard method of plasma osmolality for hydration quantification. A spectral model for total body water is developed and validated on a separate cohort of human subjects. The Raman spectroscopy device is scaled down in terms of size and complexity to collect only those spectral features used in the hydration quantification model. The result is a wearable optical device for noninvasive hydration monitoring which has been validated and tested in a military population. Raman spectroscopy is an accurate, rapid, and cost-effective method for quantitative monitoring of human hydration in real time and is an attractive alternative to methods such as sweat sensing which are only able to provide delayed markers of dehydration.

In certain embodiments, the invention utilizes a dual wavelength (e.g., about 680 nm and about 785 nm) laser source for sequential acquisition of the fingerprint and high wavenumber regions of the Raman spectrum, coupled with a spatially offset fiber optic probe for depth-sensitive measurements. To inventors' knowledge, probe-based SORS has only been implemented in the fingerprint region of the Raman spectrum, and no reports of high wavenumber SORS, or combined fingerprint and high wavenumber SORS are found. This new combination technology allows sequential acquisition of fingerprint and high wavenumber spectra by simply flipping a switch on the laser module, collecting signal from multiple depths within the sample in a single acquisition. To date, this technology has been demonstrated for quantifying water and lipid content as a function of depth in layered hydrogel-based optical phantoms, as well as for determining depth-dependent water binding profiles in tissue-based phantoms. IRB approval has been obtained for initial human subjects studies using the existing fingerprint and high wavenumber SORS system.

In certain embodiments, the combination of high wavenumber Raman spectroscopy with spatially offset Raman spectroscopy in a fiber optic probe based configuration is novel, as is the dual wavelength fingerprint and high wavenumber approach coupled with SORS in a fiber optic probe configuration.

In certain embodiments, a cart-based system capable of fingerprint and high wavenumber SORS is used for a series of experiments in layered optical phantoms to demonstrate quantitative, depth-resolved probing of water content, water binding interactions, and other biochemical components. Preliminary measurements of human skin before and after a brief hydration treatment have also been conducted, although they showed minimal change.

In certain embodiments, Monte Carlo simulation to determine the interplay between sample optical properties, excitation wavelength (about 680 nm or about 785 nm), source-detector offset, and depth of signal collection. Human subjects studies are conducted in volunteers, taking fingerprint and high wavenumber SORS before and after exercise and comparing the results to a gold standard method for hydration assessment, such as urinalysis. We also conduct more extensive studies in Vanderbilt student athletes during their training regimen following IRB approval.

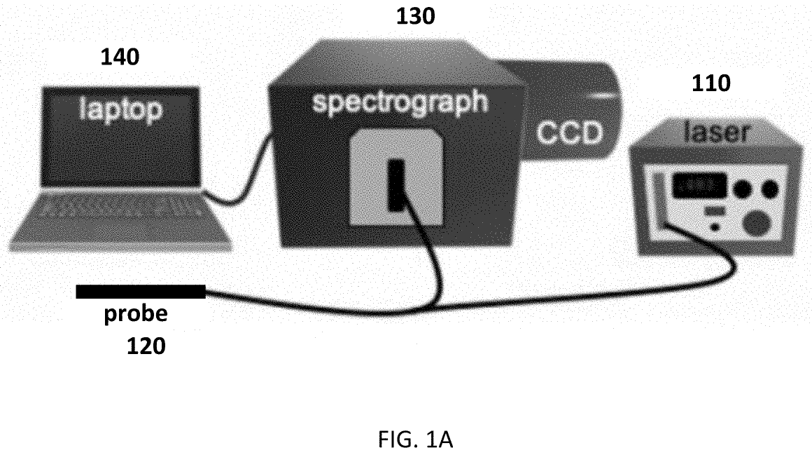

Referring to FIGS. 1A-1B, the system in one embodiment includes a light source 110 configured to operably emit light of first and second wavelengths; means 120 for delivering the emitted light to a target site 101 to excite at least one first spot at the target site, and collecting Raman scattering light scattered from the target site at a plurality of second spots in response to excitation by the light; a detector 130 coupled with said means 120 for obtaining a plurality of spatially offset Raman spectra from the collected Raman scattering light, where each spatially offset Raman spectrum is corresponding to a respective second spot of the target site, and associated with a depth of tissues at which the Raman scattering light is scattered; and a controller 140 coupled with the light source 110 and the detector 130 and configured to process the plurality of spatially offset Raman spectra so as to identify spectral features from the plurality of spatially offset Raman spectra, and assess systemic hydration from the identified spectral features. In certain embodiments, the spectral features are associated with water, electrolytes and metabolic products, and include spectral peaks in the high wavenumber region and the fingerprint region. In certain embodiments, the spectral features may further include spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

In certain embodiments, the light source comprise a dual wavelength laser module, wherein the first and second wavelengths are adapted such that when excited by the first wavelength light, the Raman scattering light corresponds to a fingerprint region; and when excited by the second wavelength light, the Raman scattering light corresponds to a high wavenumber region. In certain embodiments, the first wavelength is about 785 nm, and the second wavelength is about 680 nm. In certain embodiments, the dual wavelength laser module is configured to be operably switchable between the first wavelength and the second wavelength. In certain embodiments, the system is capable of sequential acquisition of fingerprint and high wavenumber spectra by flipping a switch on the dual wavelength laser module, and collecting signal from multiple depths within the target site in a single acquisition. In certain embodiments, each second spot is apart from the at least one first spot so as to define a source-detection (S-D) offset distance between the at least one first spot excited with the light and the second spot from which the Raman scattering light is collected. In certain embodiments, the at least one first spot comprises a plurality of first spots, and wherein the number of the plurality of first spots is smaller than the number of the plurality of second spots.

In certain embodiments, said means comprises an optical probe 120 having a working end, coupled with the light source and configured to deliver the light to the target site to excite the at least one first spot proximal to the working end, and collect from the working end Raman scattering light scattered from the target site at the plurality of second spots in response to excitation.

In certain embodiments, the optical probe comprises at least one source channel configured to deliver the light to the target site to excite the at least one first spot proximal to the working end; and a plurality of collection channels configured to collect from the working end Raman scattering light scattered from the target site at the plurality of second spots in response to excitation.

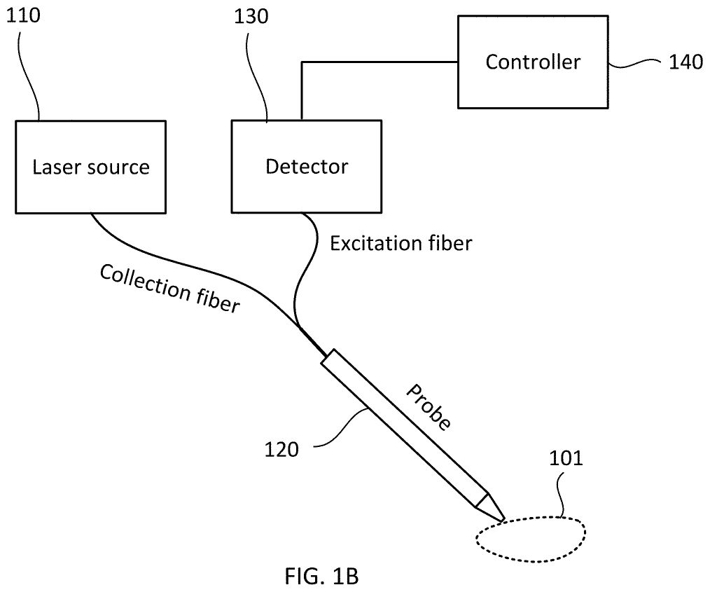

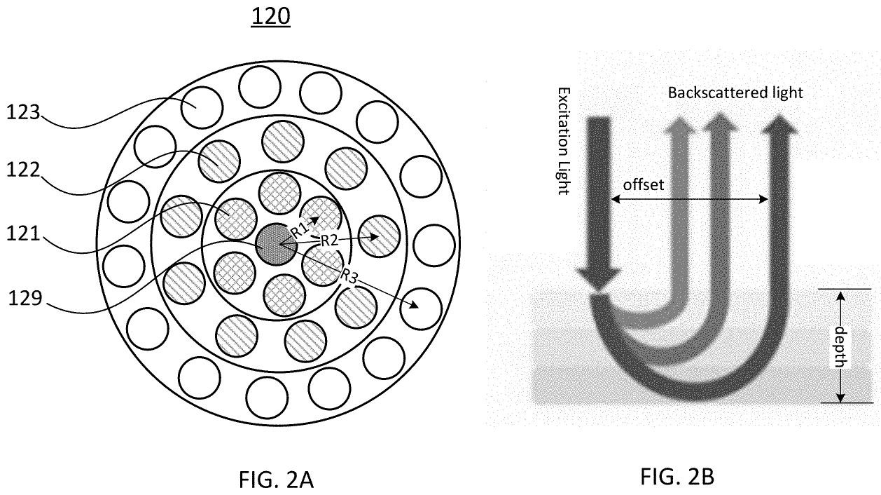

In certain embodiments, as shown in FIG. 2A, the optical probe 120 comprises at least one first fiber 129 configured to deliver the light to the target site to excite the at least one first spot proximal to the working end; and a plurality of second fibers 121, 122 and 123 configured to collect from the working end Raman scattering light scattered from the target site at the plurality of second spots in response to excitation.

In certain embodiments, the at least one first fiber and the plurality of second fibers are spatially arranged in a row or in a matrix form.

In certain embodiments, the plurality of second fibers is spatially arranged in one or more collection rings 121, 122 and 123 surrounding the at least one first fiber 129, as shown in FIG. 2A. In certain embodiments, the plurality of second fibers is spatially arranged in three collection rings originated from the at least one first fiber 129 with radii R1, R2 and R3 respectively. In certain embodiments, the optical probe is configured such that as the distance from the center of the probe increases, the number of collection fibers also increases to compensate for decreasing signal intensity with increasing offset. For example, the inner ring 121 has 6 fibers, the middle ring 122 has 9 fibers, and the outer ring 123 have 15 fibers. The inner, middle and outer rings are configured to measure hydration parameters from different depths of the skin. For example, in certain embodiments, the inner ring 121 is configured to measure hydration parameters from the epidermis of the skin, the middle ring 122 is configured to draw signal from the dermal layers of the skin, and the outer ring 123 is configured to probe sub-surface interstitial tissue hydration of the skin.

In certain embodiments, the fiber optic probe further comprises a shortpass or bandpass filter coupled to the at least one first fiber for delivering either the first wavelength light or the second wavelength light to the target site while preventing extraneous wavelengths of light from being transmitted. In certain embodiments, the shortpass or bandpass filter has a bandwidth of about 600-800 nm.

In certain embodiments, the fiber optic probe further comprises a blocking filter such as a longpass filter coupled to the plurality of second fibers for preventing backscattered excitation light from being collected. In certain embodiments, the longpass filter has an cut-on wavelength of about 800 nm.

In certain embodiments, the detector comprises a spectrograph and/or a sensing member such as a camera. In certain embodiments, the sensing member comprises at least one charge-coupled device (CCD) camera, and/or at least one complementary metal oxide semiconductor (CMOS) camera, and/or at least one photodiode.

In certain embodiments, the controller is configured to correlate, using a Spearman correlation, the identified peaks and spectral ratios for each depth of collection corresponding to the inner, outer and middle rings of the optical probe with plasma osmolality-based assessment of hydration level, so as to determine the optimal depth of measurement for hydration assessment based on these correlation values.

In certain embodiments, the controller is configured to analyze the changes associated with hydration level using multivariate statistical and machine learning approaches. In certain embodiments, the analysis utilizes generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, X is a matrix containing independent variables of BMI, age and temperature, p is a vector containing weight coefficients of the independent variables, and E is a residual error in the GLM, wherein a linear least squares regression is performed to choosep coefficients such that E is minimized.

In certain embodiments, the controller is configured to perform Voigtian decomposition of the high wavenumber region of the Raman spectra into the spectral peaks including five component water peaks and an N-H peak in the same region, so as to analyze the distribution of fully-bound, partially bound, and free water molecules in the target site.

In another aspect, the invention relates to a method for real-time assessment of systemic hydration, comprising exciting a target site at at least one first spot with light of first and second wavelengths; collecting Raman scattering light from the target site at a plurality of second spots, respectively, in response to illumination by the light, wherein each second spot is apart from the at least one first spot so as to define a source-detection (S-D) offset distance between the at least one first spot excited with the light and the second spot from which the Raman scattering light is collected; obtaining the plurality of spatially offset Raman spectra from the collected Raman scattering light, wherein each spatially offset Raman spectrum is corresponding to a respective second spot of the target site, and associated with a depth of tissues at which the Raman light is scattered; identifying spectral features from the plurality of spatially offset Raman spectra; and determining systemic hydration from the identified spectral features.

In certain embodiments, the first and second wavelengths are adapted such that when excited by the first wavelength light, the Raman scattering light corresponds to a fingerprint region; and when excited by the second wavelength light, the Raman scattering light corresponds to a high wavenumber region. In certain embodiments, the first wavelength is about 785 nm, and the second wavelength is in a range of about 660-700 nm.

In certain embodiments, the exciting step comprises exciting the target site at the at least one first spot with the first wavelength light and the second wavelength light sequentially, so that the fingerprint and high wavenumber spectra are sequentially acquired, and signals from multiple depths within the target site are collected in a single acquisition.

In certain embodiments, the at least one first spot comprises a plurality of first spots, and wherein the number of the plurality of first spots is smaller than the number of the plurality of second spots.

In certain embodiments, the exciting and collecting steps are performed with an optical probe having a working end.

In certain embodiments, the optical probe comprises: at least one source channel configured to deliver the light to the target site to excite the at least one first spot proximal to the working end; and a plurality of collection channels configured to collect from the working end Raman scattering light scattered from the target site at the plurality of second spots in response to excitation.

In certain embodiments, the optical probe comprises: at least one first fiber configured to deliver the light to the target site to excite the at least one first spot proximal to the working end; and a plurality of second fibers configured to collect from the working end Raman scattering light scattered from the target site at the plurality of second spots in response to excitation.

In certain embodiments, the at least one first fiber and the plurality of second fibers are spatially arranged in a row or in a matrix form.

In certain embodiments, the plurality of second fibers is spatially arranged in one or more collection rings surrounding the at least one first fiber.

In certain embodiments, the plurality of second fibers is spatially arranged in three collection rings originated from the at least one first fiber with radii R1, R2 and R3 respectively.

In certain embodiments, the optical probe is configured such that as the distance from the center of the probe increases, the number of collection fibers also increases to compensate for decreasing signal intensity with increasing offset.

In certain embodiments, the inner ring is configured to measure hydration parameters from the epidermis of the skin, the middle ring is configured to draw signal from the dermal layers of the skin, and the outer ring is configured to probe sub-surface interstitial tissue hydration of the skin.

In certain embodiments, the fiber optic probe further comprises a shortpass or bandpass filter coupled to the at least one first fiber for delivering either the first wavelength light or the second wavelength light to the target site while preventing extraneous wavelengths of light from being transmitted.

In certain embodiments, the shortpass or bandpass filter has a bandwidth of about 600-800 nm.

In certain embodiments, the fiber optic probe further comprises a blocking filter such as a longpass filter coupled to the plurality of second fibers for preventing backscattered excitation light from being collected.

In certain embodiments, the longpass filter has an cut-on wavelength of about 800 nm. In certain embodiments, the obtaining step is performed with a detector comprising a spectrograph and/or a sensing member.

In certain embodiments, the sensing member comprises at least one charge-coupled device (CCD), at least one complementary metal oxide semiconductor (CMOS), and/or at least one photodiode.

In certain embodiments, the spectral features are associated with water, electrolytes and metabolic products, and include spectral peaks in the high wavenumber region and the fingerprint region.

In certain embodiments, the method further comprises correlating, using a Spearman correlation, the identified peaks and spectral ratios for each depth of collection corresponding to the inner, outer and middle rings of the optical probe with plasma osmolality-based assessment of hydration level, so as to determine the optimal depth of measurement for hydration assessment based on these correlation values.

In certain embodiments, the method further comprises analyzing the changes associated with hydration level using multivariate statistical and machine learning approaches.

In certain embodiments, the analyzing step is performed with generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, Xis a matrix containing independent variables of BMI, age and temperature, p is a vector containing weight coefficients of the independent variables, and E is a residual error in the GLM, wherein a linear least squares regression is performed to choose p coefficients such that E is minimized.

In certain embodiments, the method further comprises performing Voigtian decomposition of the high wavenumber region of the Raman spectra into the spectral peaks including five component water peaks and an N-H peak in the same region, so as to analyze the distribution of fully-bound, partially bound, and free water molecules in the target site.

In one embodiment, the spectral features further includes spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

In yet another aspect, the invention relates to a device using Raman spectral features for real-time assessment of systemic hydration of a subject in biomedical applications. The Raman spectral features are associated with water, electrolytes and metabolic products and include spectral peaks in the high wavenumber region and the fingerprint region. The device in one embodiment comprises a light source configured to emit light to excite a target site in the subject with an excitation wavelength that is determined based on wavenumber locations of the spectral peaks to be detected; a filter configured, for each feature to be detected, to pass Raman scattering light of the wavenumber range at which that feature falls with respect to the given excitation wavelength, wherein the Raman scattering light is scattered from the target site in response to excitation by the light; a detector configured to detect signals of the Raman scattering light passing through the filter; and a controller configured to operate the light source and process the detected signals by the detector to determine systemic hydration of a subject in real time.

In certain embodiments, the light source comprises a VCSEL-technology based single monochromatic light source.

In certain embodiments, the device further comprises dichroic mirrors (DMs) configured to direct the path of the emitted light.

In certain embodiments, the device further comprises an alert configured to generate an audible signal and/or a visible signal whenever the measured hydration level falls below what is determined to be normal for the subject.

In certain embodiments, the filter comprises at least one linear variable filter (LVF), and/or at least one high-throughput narrow bandpass filter.

In certain embodiments, the detector comprises a CCD camera, a CMOS camera, and/or photodiode.

In certain embodiments, the detector comprises a an avalanche photodiode (APD), and/or a cooled linear diode array.

In certain embodiments, the device further comprises one or more slit apertures located between the filter and the detector.

In certain embodiments, the device is configured to accurately and noninvasively measure systemic hydration in a user and to send the measured results to one or more external devices for real-time remote monitoring, and/or data collection and storage.

In certain embodiments, the device is configured to be wearable and portable.

The main advantage of this technology over existing SORS or dual wavelength systems lies in the capability for quantitative, depth-sensitive analysis of water content and water-tissue interactions. We have disclosed this technology for systemic hydration monitoring in populations at high risk for dehydration—this may include the military, athletes, the elderly, and neonates. This technology may have applications in other fields which benefit from depth-dependent water content information along with other biochemical signatures, such as food science or materials science.

These and other aspects of the present invention are further described below. Without intent to limit the scope of the invention, examples according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE 1

### Hydration Characterization Using Fingerprint and High Wavenumber Raman Spectroscopy In Vivo

Initial studies of human hydration are conducted at Vanderbilt University. A preliminary study of individuals before and after exercise is performed to optimize measurement parameters, characterize hydration-associated changes, and estimate the required sample size for subsequent human studies. A more comprehensive study under prescribed conditions is then conducted in student athletes at Vanderbilt University. Raman measurements of human subjects are taken before and after training to accurately determine water and electrolyte content in tissue during both the hydrated and dehydrated states. These measurements are also taken from various locations on the body in order to determine which site yields a measure that is most representative of the total body water. Total body water is compared to values determined using plasma osmolality, the gold standard for assessing hydration status, and bioelectrical impedance spectroscopy, which is commonly used in the field to evaluate hydration level.

Rationale: In order to develop a robust method for quantifying systemic hydration using Raman spectroscopy, full fingerprint and high wavenumber Raman spectra must be collected first from human subjects at various levels of hydration. A blood draw is performed at the same time points as Raman measurements to determine total body water using the current gold standard method. The collection of this data under controlled conditions designed to mimic those which might be encountered during military training and battle facilitates accurate quantification of hydration using Raman spectroscopy.

Instrumentation: The Raman system employed for this study is shown in FIGS. 1A-1B. This system includes a dual wavelength diode laser with outputs at 680 and 785 nm manufactured by Innovative Photonics Solutions, a f/1.8i high throughput imaging spectrograph (Kaiser Holospec), a thermoelectrically cooled, deep depletion charge coupled device (Princeton Instruments Pixis 400), and a custom designed fiber optic probe. The system is controlled by a laptop and custom LabView software. The fiber optic probe is designed in and constructed in collaboration with EmVision, LLC and includes a single excitation fiber with a 600-800 nm bandpass filter at the distal dip, surrounded by three rings of collection fibers at 1 mm, 2 mm, and 3 mm offsets from the center of the excitation fiber, respectively. An annular 800 nm longpass filter is integrated into the probe at the distal tip of the collection fibers. As the distance from the center of the probe increases, the number of collection fibers also increases to compensate for decreasing signal intensity with increasing offset, as shown in FIG. 2A. This SORS probe configuration enables the collection of Raman signal from three different depths in tissue and the selection of the optimal depth for a hydration monitoring measurement. The probing depth of each collection ring has been estimated based on measurements taken in tissue-mimicking phantoms (Table 1), although the exact depth from which signal is collected depends on the unique optical properties of the sample. For reference, the epidermis comprises approximately the first 100 microns of skin thickness, while the total skin thickness is approximately 1-2 mm, although this can vary widely based on the location on the body. Thus, based on our calculations, ring 1 measures hydration parameters from the epidermis, ring 2 draws signal from the dermal layers of the skin and ring 3 probes sub-surface interstitial tissue hydration.

TABLE 1
Estimated probing depth for each ring of the SORS probe at about 785
and 680 nm excitation

| | Inner Ring (1 mm) | Middle Ring (2 nm) | Outer Ring (3 nm) |
|---|---|---|---|
| 785 nm | 0.6940 mm | 1.8485 mm | 6.9670 mm |
| 680 nm | 0.7107 mm | 1.4615 mm | 3.7838 mm |

Sample Size Calculation: There are no prior studies using Raman spectroscopy to evaluate systemic hydration levels in vivo. In this exemplary example, a sample size of 50 subjects is utilized for this pilot study in order to characterize the spectral changes that correspond to normal, high, and low hydration levels. Once preliminary human data is available, a power calculation is performed, and the target recruitment numbers are adjusted accordingly.

Experimental Protocol and Human Subjects: Preliminary data collection for this study is performed at Vanderbilt University in collaboration with the Department of Sports Medicine. After obtaining the proper IRB approval, student athletes at Vanderbilt aged 18 and over are enrolled in the study using informed written consent. The goal of this portion of the study is to collect data from individuals undergoing exertion under a variety of controlled conditions similar to those which might be experienced as part of military training in order to develop an accurate and robust model for hydration quantification. In an effort standardize the study measurements, the prescribed workouts, Raman measurements, and hydration protocols are performed first thing in the morning prior to eating breakfast. A baseline measurement of each athlete is taken prior to the start of exercise with the SORS system described above. Thermal modeling previously performed has demonstrated that the excitation parameters used does not cause significant heating or thermal damage to skin tissue of various pigmentations. Further detail regarding the protections of human subjects that are in place throughout the completion of this study can be found in the document entitled “Human Subject Recruitment and Safety Procedures.”

Upon enrollment in the study, basic medical data are collected for each participant, including age, height, weight, and medical history. Each athlete in the study is asked to undergo both aerobic exercise (i.e., yoga, pilates, etc.) and inaerobic exercise (cardio workouts such as running) of 30 minute, 1 hour, and 2 hour duration under normal room temperature (approximately 22° C.), high temperature (35° C.), and low temperature (0° C.). Each athlete is also assigned to one of four rehydration protocols in combination with their workout: hydrate as normal during the workout (amount of water consumed is recorded), consume 1 liter of water immediately following the workout followed by no additional hydration for the remaining 3 hour measurement period, consume 1 liter of water each hour for the 3 hour measurement period, or do not consume any water during the three hour measurement period. Table 2 summarizes the study conditions to which each athlete is exposed. Raman spectral measurements are taken every 15 minutes during the workout and during the three hours following the workout. In order to correlate Raman spectral data with plasma osmolality, the gold standard method for hydration assessment, a blood draw is performed at the time of each Raman measurement, except for those measurements taken during the workout. A sports medicine clinician monitors the athletes throughout the prescribed workout and rehydration protocol, and should any participant begin to exhibit negative side effects, they immediately receives the appropriate care.

TABLE 2
Exercise and rehydration conditions that each athlete undergo during the
experimental procedure

| | Exercise | Exercise | |
|---|---|---|---|
| Exercise Type | Duration | Condition | Rehydration Protocol |
| Aerobic | 30 minutes | 0° C. | Hydrate normally |
| Anaerobic | 1 hour | 22° C. | 1 liter water immediately after |
| | | | exercise |
| | 2 hours | 35° C. | 1 liter water immediately each |
| | | | hour for 3 hours |
| | | | Do not hydrate until 3 hours after |
| | | | exercise |

The acquired spectra are calibrated, fluorescence subtracted, and processed as previously described. The spectra for each participant under each combination of workout and rehydration conditions are plotted, and spectral features which differ across each condition are identified.

### EXAMPLE 2

### A Model for Quantifying Water, Electrolytes, and Other Relevant Biomarkers

Analysis of the high wavenumber water band as well as features from the fingerprint region is fed into a machine learning algorithm to develop an optimized model that can determine a person's hydration level based on select spectral features. This model is compared to the gold standard for performance and is then applied to predict the hydration level of a separate validation cohort of patients under various conditions. The performance of the SORS for hydration quantification is determined based on the gold standard method and compared to existing portable techniques such as bioimpedance. To evaluate the validity of this model, airmen who are between deployments or undergoing training at Wright Patterson Air Force Base are recruited and measured, in a collaboration with the 711th Human Performance Wing and the Uniformed Services University of the Health Sciences in the Family Sports Medicine Department. The hydration level of airmen is predicted using the aforementioned model and is compared to the gold standard to determine the model accuracy.

Rationale: An accurate and robust model for quantifying hydration in individuals such as military personnel must be able to account for the wide range of conditions that the individuals experience, as well as person-to-person variability. Therefore, we develop a model which incorporates multiple spectral features from fingerprint as well as high wavenumber Raman features including those associated with water, electrolytes, and metabolic products, in addition to accounting for individual variables such as age, weight, body mass index as well as environmental parameters such as humidity and temperature. Multivariate statistical as well as machine learning approaches are used to develop a robust model for quantifying hydration. To ensure that this technology performs effectively in the population for which it is designed, validation is performed on service members between deployments at Wright Patterson Air Force Base. The model results are compared to gold standard plasma osmolality to estimate performance and refine the model as needed.

Spectral Analysis and Predictive Model: Voigtian decomposition of the high wavenumber water band is performed to analyze the distribution of fully-bound, partially bound, and free water molecules in the tissue. Previous work has demonstrated that this ratio shifts in response to changing electrolyte concentrations, which is a vital component for understanding systemic hydration. Other peaks which change as a function of hydration level as determined by plasma osmolality are identified and analyzed, either as individual peak amplitudes or as peak ratios. Using a Spearman correlation, the identified peaks and spectral ratios for each depth of collection (corresponding to the inner, outer and middle rings of the SORS probe) are correlated with plasma osmolality-based assessment of hydration level. Based on these correlation values, the optimal depth of measurement for hydration assessment is determined.

The changes associated with hydration level is analyzed using multivariate statistical and machine learning approaches. We utilize generalized linear models (GLM), a method capable of incorporating results from Raman spectra, as well as participant factors such as body mass index (BMI). Generalized linear models follow the simple linear equation: Y=pX+E, where Y is a vector containing the dependent variable (in this case, Raman spectra), X is a matrix containing independent variable(s) (age, BMI, temperature), p is a vector containing coefficients of the independent variables (weights), and E is the residual error in the model. A linear least squares regression is then performed to choosep coefficients such that E is minimized. The impact of participant variables such as gender, age, height and weight on Raman spectra are quantified using this approach, and those variables which are found to have a significant effect are incorporated into the final predictive model.

The significant trends in peaks and peak ratios are used to develop a predictive model using machine learning. This model incorporates up to five spectral features across both the fingerprint and high wavenumber regions as well as include other correlative parameters such as environmental temperature, humidity, etc. and individual demographics such as age, BMI, etc., to predict a person's hydration level and categorization as either hyperhydrated, euhydrated, or dehydrated. The validity of this model is assessed using leave-one-person-out and k-fold cross validation. This model is incorporated in software with the system so that model validation may be performed in real-time.

Performance Assessment of Predictive Model: To evaluate how well the developed model performs for individuals such as military personnel, measurements are acquired from twenty military personnel on base between deployment or undergoing training at Wright Patterson Air Force Base in a collaboration with the Air Force Research Lab at this location, and the proper IRB approval and informed written consents is obtained prior to the start of testing. Measurements are acquired before, during and after training exercises using the Raman system as described in EXAMPLE 1. Variables such as the service member's age, gender, height, and weight are recorded, and although water consumption and the content of the training regimen are not regulated as part of the experimental protocol, they are recorded and used to inform data analysis. Blood draws for plasma osmolality measurements are performed before and after training as this is currently being implemented as part of other protocols at the AFRL. The spectra acquired are used as input to the predictive model, and the hydration value determined by the model in real time is compared with the plasma osmolality-based metric to quantify the accuracy of the Raman-based approach. Based on the results, the model is refined as needed and retested.

### EXAMPLE 3

### A Portable SORS-Based Monitor to Quantify Hydration Based on Select Wavelengths

By measuring the entire Raman spectrum, current RS devices are often more information-rich than is necessary to accurately detect the relevant biochemical features. Several handheld spectrometers in the market do not consider interfering background fluorescence present in tissues and are therefore not useable for biomedical Raman spectroscopy. By tailoring a Raman spectrometer to detect only certain spectral regions of interest that is indicative of change in the sample under study, the cost and complexity of both the instrument and acquired data can be significantly reduced. The device includes a diode laser source, bandpass filter, and avalanche photodiode as well as focusing optics. The spectral features used in EXAMPLE 2 as well as design consideration such as size, durability, and cost guide the design and prototyping of this device.

Rationale: In order to be deployable in military settings, which may include training facilities, battlefields, and remote locations, a hydration monitoring device must be portable/wearable, durable, easy to use, and communicate accurate real-time results in a secure manner. In some embodiments, a traditional Raman spectroscopy system, which can be bulky and challenging to operate for an untrained user, is scaled down to provide only that information which is needed for accurate hydration prediction, where the entire Raman spectrum is not required, only those features identified by the model developed in EXAMPLE 2 are critical.

The concept of developing a compact Raman spectrometer is not new. Multiple commercial handheld systems mainly targeted towards identifying hazardous substances exist and these exhibit many desired qualities such as being battery powered, highly stable, and easily operable systems. However, all existing commercial handheld instruments use CCD/CMOS cameras or photodiode arrays paired with dispersion elements to generate a full Raman spectrum. Further, due to the need for miniaturization and the purpose of these devices, the quantum efficiency and noise characteristics of these detectors do not allow detection of the weak RS signals generated by tissues. None of these devices measure the fluorescence background present in tissues that needs to be eliminated before notable tissue Raman features may be extracted. Some vendors have “compact” Raman spectrometers with more sensitive detectors that are suggested for biomedical use. However, while these are portable, they are not wearable and their performance remains sub-optimal for tissue Raman measurements. On the other hand, clinically viable RS instruments typically use deep depletion back illuminated NIR optimized CCD detectors which have significant power and thermo-electric cooling requirements, coupled to fiber optimized spectrographs which result in devices that cannot be easily miniaturized. To date, no wearable Raman devices exist for tissue Raman measurements.

Instrument Development: By measuring the entire Raman spectrum, current RS devices are often more information-rich than is necessary to accurately detect the relevant biochemical features. In certain embodiment, by tailoring a Raman spectrometer to detect only certain spectral regions of interest as determined by the sample under study, the cost and complexity of both the instrument and acquired data can be significantly reduced. The novel instrument detects only those wavelengths corresponding to Raman spectral features that are utilized in the predictive model for hydration assessment, rather than the entire fingerprint and high wavenumber spectrum. An additional 3 bands measure the fluorescence background to improve the accuracy of the device. The minimum number of features needed are determined.

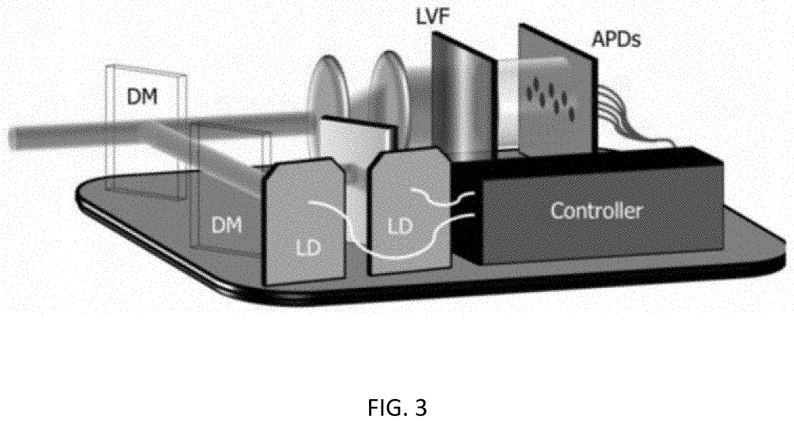

In the development of the first wearable Raman device, a single monochromatic light source based on VCSEL-technology is utilized; its exact wavelength is determined based on the wavenumber locations of the peaks we wish to detect. For each feature to be detected, a linear variable filter (LVF) is tuned to pass the wavenumber range at which that feature falls with respect to the given excitation wavelength. The light which passes through the filter is detected by an avalanche photodiode (APD), which is much smaller and less expensive than the CCDs typically used in Raman spectroscopy systems. An analog-to-digital data acquisition card can be used to read out the intensity of light detected by the APD, and the values for the APD at each wavelength are used as input to the predictive model to determine a person's hydration level. FIG. 3 shows a general schematic of this system, which also includes dichroic mirrors (DMs) to direct the path of the light and a controller to operate the laser diode (LD) and read out the APD signal. The on-board computer feeds the recorded information into the model and an audible signal sounds whenever the measured hydration falls below what is determined to be “normal” for a given test subject. Ultimately, the device is optimized to fit within a small portable form factor that can be easily worn as a patch or band containing the Raman sensor components in contact with the skin, tethered to a small controller which collects and stores the data.

Phantom Validation: Initial validation of this portable Raman device takes place at Vanderbilt using gel-based tissue-mimicking phantoms. Phantoms are formulated using variable ratios of gelatin, water, aluminum oxide (scattering agent), and intralipid (scattering agent and generator of strong Raman signal). Spectra are collected for phantoms ranging from 50 to 90 percent water. The configuration of electronics in the device, power of the light source, acquisition time, cut on/cut off wavelength of the filters, and other parameters are optimized to provide the highest possible signal-to-noise ratio (SNR). The measured intensities at each wavelength are used as input to the predictive model in order to assess the model performance in conjunction with the portable Raman device. Adjustments to the model and/or additional filters and features are incorporated as needed.

Testing and Validation in Human Subjects: Once optimized at Vanderbilt, the wearable hydration sensor is tested for use in 20 military service members at Wright Patterson Air Force Base. The device is used to provide continuous monitoring of the participants during training exercises. A blood draw is performed before and after the training exercises to provide a plasma osmolality-based assessment, the accuracy of the wearable sensor for hydration monitoring is determined by comparison to this metric. Additionally, the study participants are surveyed regarding their experience using the device. The accuracy of this technology compared to the gold standard and the ease of use by military personnel is taken into consideration for future iterations of the device.

As a new design that utilizes sparsely-sample Raman spectral information, issues regarding detection sensitivity and spectral resolution must be addressed. The incorporation of source and detector elements is mediated by the instrument SNR performance. Although the need is not anticipated, if the system does not produce the desired SNR due to low sensitivity or high noise for the bands of interest in less than 10 seconds of exposure time, a cooled linear diode array is used for detection. Furthermore, if the spectral resolution is not sufficient for the bands desired, slit apertures may be included between the LVF and APDs or the LVF may be replaced with high-throughput narrow bandpass filters, or both. These design considerations still retain the sparse-sampling approach needed for a robust and versatile wearable instrument.

The prototype device is capable of accurately and noninvasively measuring systemic hydration in military personnel and sending the results for real-time remote monitoring. This represents a significant step towards being able to identify dehydrated military personnel in the field before their performance begins to be compromised, a critical need area for which no technology currently exists.

### EXAMPLE 4

### Preliminary Data

In the exemplary example, a dual excitation wavelength system for combined fingerprint and high wavenumber Raman spectroscopy, as well as its validation for water quantification in optical phantoms are disclosed. The system employs excitation at 680 nm for collection of high wavenumber spectral information, and excitation at 785 to enable fingerprint spectral acquisition. This configuration is easier to use, more portable, and more cost effective than previous iterations of combined fingerprint and high wavenumber Raman spectroscopy systems. The dual wavelength laser is coupled to a fiber optic probe with filtering that allows either the 680 or 785 nm excitation light to pass through the center excitation fiber, while Raman scattered photons corresponding to either the fingerprint or high wavenumber region pass through the seven surrounding collection fibers which are coupled to an imaging spectrograph and thermoelectrically cooled, deep depletion CCD. Switching between the fingerprint and high wavenumber region is accomplished by flipping a switch at the front of the laser module.

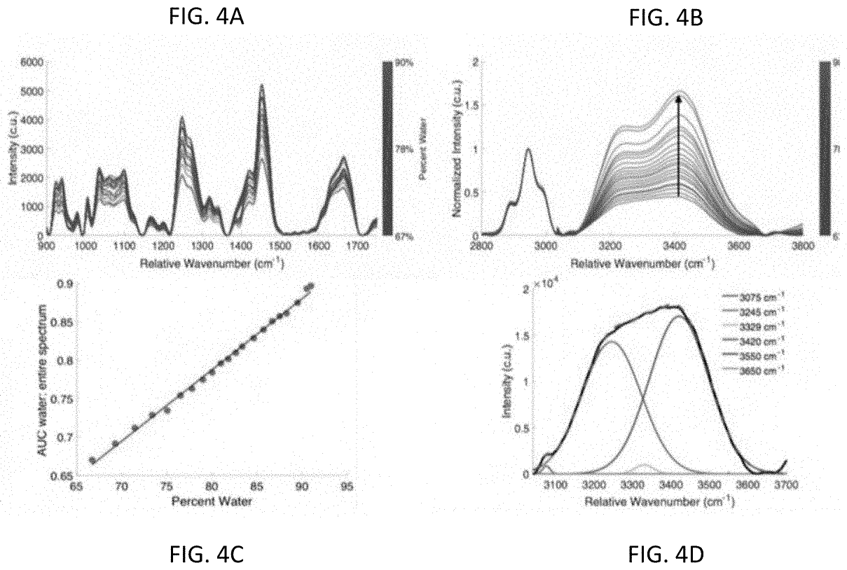

The ability of this system to quantify changes in water content is validated using tissue-mimicking phantoms, which includes varying concentrations of gelatin and water. Fingerprint and high wavenumber spectra are acquired from each phantom and analyzed as a function of percent water. As shown in FIG. 4A, spectral features in the fingerprint region, which are predominantly associated with proteins and collagen, the major components of gelatin, decrease in intensity as the percent water in the phantoms increases. Correspondingly, in the high wavenumber region, the amplitude of the water peak increases as the percent water in the phantom increases, as shown in FIG. 4B. In an effort to quantify this change and develop a spectral metric which can be used to estimate the percent water, several high wavenumber peak ratios are analyzed. The ratio of the area under the curve (AUC) of the water peak (3035 to 3680 cm⁻¹) to the AUC of the entire high wavenumber spectrum (2850 to 3680 cm⁻¹) has a roughly linear relationship with respect to the percent water as shown in FIG. 4C. This demonstrates the ability to quantitatively assess water content in a sample based on Raman spectral metrics and shows feasibility of using combined fingerprint and high wavenumber Raman spectroscopy to evaluate hydration at the systemic level. Further quantitative analysis of water content is performed by decomposing the high wavenumber into five component water peaks and an N-H peak in the same region using a Voigtian fit. This decomposition approach enables comparison of fully and partially bound water in tissue, providing useful information about complex water-tissue interactions. An example of this fitting performed on a gelatin phantom spectrum is shown in FIG. 4D. This spectral analysis forms the basis for the proposed software development to enable quantification of systemic hydration.

This hydration approach in vivo in a model system is tested as part of a separate study which seeks to understand biochemical change in the cervix during pregnancy. Combined fingerprint and high wavenumber Raman spectra are acquired in vivo from the cervices of non-pregnant mice and mice on the final day of pregnancy (day 19). The fingerprint region demonstrated decreased features associated with collagen and lipids in the day 19 mouse compared to the non-pregnant (non-gravid) mouse. The high wavenumber region showed an increase in the water peak amplitude and more partially bound water relative to fully bound water compared to the non-gravid mouse, validating a phenomenon which has previously only been measured ex vivo. This study showcases the utility of the dual wavelength combined fingerprint and high wavenumber system for in vivo application and assessment of tissue hydration. The work disclosed herein is used to develop, test, and optimize a wearable Raman spectroscopy device for real-time assessment of systemic hydration in biomedical applications.

### EXAMPLE 5

### Combined Fingerprint and High Wavenumber Spatially Offset Raman Spectroscopy for Depth-Resolved Hydration and Biochemical Assessment

Raman spectroscopy (RS) probes the vibrational modes of molecules and yields a sample-specific molecular fingerprint (FP), from which biochemical composition can be inferred. The vast majority of Raman spectroscopic studies for biomedical applications have focused on the low wavenumber or fingerprint region (500-1800 cm⁻¹), as it is information-rich including signatures associated with proteins, lipids, DNA, and blood. However, analysis of the high wavenumber (HW) region (2500-3800 cm⁻¹) provides detailed information regarding the molecular dynamics of water which is not possible when studying the fingerprint region alone. Water molecules produce a broad and high intensity band centered at approximately 3350 cm⁻¹, which can be used to discriminate between fully hydrogen bound, partially hydrogen bound, and unbound water molecules based on deconvolution of the water band into its five component peaks. The fingerprint and high wavenumber regions together provide a more complete biochemical picture of the sample composition.

As disclosed above, the RS system is capable of acquiring both fingerprint (FP) and high wavenumber (HW) Raman spectra using a single laser unit containing 680 and 785 nm excitation wavelength laser diodes. This configuration allows both the fingerprint (at 785 nm excitation) and the high wavenumber region (at 680 nm excitation) to fall within the CCD's wavelength range of optimal sensitivity. In this exemplary study, we present on the development, characterization, and implementation of dual excitation wavelength SORS for depth-resolved fingerprint and high wavenumber Raman spectroscopy. The SORS is a form of RS in which the excitation source and detector are separated by some spatial offset. As the distance between the source and detector increases, on average the photons have travelled deeper into the sample before undergoing scattering events and returning to the detector, thus enabling the collection of biochemical information from deeper layers of the material. Combining the SORS with dual region RS is a novel approach to the biochemical evaluation of materials and tissue. Depth-resolved hydration assessment along with the biochemical markers present in the fingerprint region is of value for numerous applications, such as cancer characterization and detection, probing biochemical change in the pregnant cervix, and exploring the penetration of various moisturizing agents and disease conditions in the skin.

Instrumentation: Raman spectra are acquired using a fiber-optic probe-based Raman spectroscopy system. The fiber optic probe includes a single 300-micron diameter excitation fiber surrounded by three rings of collection fibers with offsets of 1 mm, 2 mm, and 3 mm, respectively, as measured from the center of the excitation fiber to the center of any given collection fiber in that ring. All collection fibers are 200 microns in diameter. The inner ring includes 6 collection fibers, the middle ring has 9 collection fibers, and the outer ring has 15 collection fibers, as shown in FIG. 2A. The collection fibers are further divided into four quadrants which can be separated based on their orientation upon entering the spectrograph, enabling further spatial discrimination for heterogeneous samples. The excitation fiber connects via an FC/PC connector to a combined 680 and 785 nm diode laser (Innovative Photonics Solutions) which has been previously described. The collection fibers of the probe deliver the backscattered light to an imaging spectrograph (Princeton Instruments Acton LS-785) which then disperses the light onto a thermoelectrically cooled, deep depletion CCD camera (Pixis 400, Princeton Instruments). The system is controlled with a laptop and custom LabView program and has a spectral resolution of 5 cm⁻¹.

Depth Characterization: A layered synthetic phantom including upper and lower layers each with strong but distinct Raman signals is created, with the upper layer including a variable number of parafilm layers, each 130 microns in thickness, and the lower layer including a polystyrene block. The SORS probe is placed in contact with the polystyrene layer and spectra are acquired using 80 mW excitation power and a 30 second exposure time at both 785 nm and 680 nm excitation. A single parafilm layer of 130 micron thickness is placed over the polystyrene layer and spectra are acquired in the same manner at both excitation wavelengths. This procedure is repeated until a total of 40 parafilm layers (total thickness 5.2 mm) had been placed atop the polystyrene layer and measured. Finally, a measurement of just the 40 parafilm layers is acquired. The penetration depth for each ring of collection fibers at 680 and 795 nm excitation is calculated based on the contribution of polystyrene as determined using a non-negative least squares fit of pure polystyrene and pure parafilm layers to the spectra measured from layered phantoms.

Optical Phantom Experiments: Hydrogel phantoms containing a bottom layer of variable water or lipid content overlaid with upper layers of constant concentration are constructed. The variable water bottom layers including 8 ml of intralipid to introduce scattering and provide a distinct Raman signal, 1.95 g of aluminum oxide to further increase scattering, along with gelatin and water to a final volume of 40 ml. The amounts of water and gelatin in are adjusted to create phantoms of 70%, 65%, 60%, 55%, and 50% (weight per volume concentration) water. Likewise, the amounts of intralipid and gelatin in a separate set of phantoms are adjusted to create phantoms of 0%, 10%, 20%, 30%, and 40% intralipid. The gelatin solution is mixed over 93° C. heat until the gelatin is dissolved and a homogenous mixture is achieved. The mixture is then poured into a rectangular mold to create a gel with approximately 5 mm thickness. Agarose layers of 500 micron thickness including 2% agarose with aluminum oxide for scattering are also created. Both the gelatin and agarose layers are allowed to cool at approximately 4° C. prior to measurements. For each concentration, spectra are acquired at 80 mW incident power, 10 second acquisition, at both 785 nm and 680 nm excitation. This procedure is repeated for zero to five agarose upper layers (2.5 mm total thickness), and the entire process is repeated for each concentration. To determine the ability to detect a small, localized inclusion with strong Raman signal, a polystyrene bead is embedded in a 5 mm thickness block of agarose, and parafilm layers are placed over top while spectra are collected in the manner described above. Monte Carlo modeling is used to simulate these phantom configurations and to explore the interplay between optical properties, spatial offset, and probing depth.

Results and Discussion: While high wavenumber Raman spectroscopy has proven useful for analysis of tissue protein, lipid, and particularly water content, to our knowledge SORS has been limited to the fingerprint region. This presents an opportunity for depth-resolved hydration analysis in addition to the rich biochemical specificity of the fingerprint region.

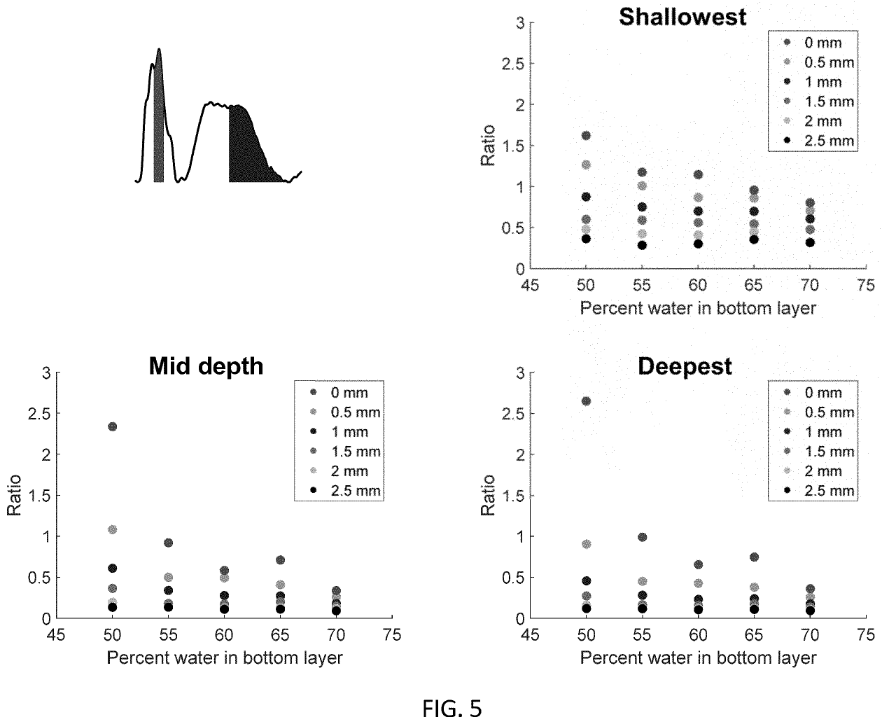

In order to determine the depth at which each ring of collection fibers probes, synthetic phantoms with variable numbers of upper layers are measured. Based on the decrease in intensity of peaks associated with the bottom (polystyrene) layer as the number of upper (parafilm) layers increased, the probing depth of each ring at 680 and 785 nm excitation is determined. The inner and most shallow ring achieved a probing depth of 1.4731 mm at 785 nm and 1.0046 mm at 680 nm, while the middle ring achieved depths of 1.7073 mm at 785 nm excitation and 1.0931 mm at 680 nm, and the outer and deepest probing ring acquired signal from depths of 2.0248 at 785 nm and 1.3898 mm at 680 nm. Gel-based tissue-mimicking optical phantoms are measured in order to evaluate the capability of this system to quantify changes in tissue hydration and lipid content in depth. The high wavenumber region is utilized for hydration analysis while fingerprint spectral features are analyzed for phantoms varying in lipid content. The ratio of the high wavenumber peak attributed to C-H stretching in gelatin, located at 2930 cm⁻¹, to the left side of the high wavenumber water band, located at 3250 cm⁻¹, shows an inverse relationship with the percent water in the bottom layer of variable water phantoms. All three rings show the same overall relationship, although the outer ring demonstrates a greater difference between the maximum and minimum number of upper layers, especially for phantoms with lower water content. The ratio of the area under the high wavenumber C-H stretch region from 2910 to 2985 cm⁻¹ to the area under the water band from 3350 to 3550 cm⁻¹ show a similar inverse relationship with the water content in phantoms, and further shows increased separation between the ratio as the number of upper layers increases, particularly for phantoms with lower water content, as shown in FIG. 5. A similar relationship is observed in variable lipid phantoms, where several fingerprint spectral ratios are plotted against the percent lipid and showed a high correlation. When a polystyrene bead is embedded in an agarose block, a depth-dependent decrease in polystyrene-associated fingerprint and high wavenumber features is observed as a function of both increasing thickness of the upper parafilm layer as well as increasing lateral distance between the probe and the polystyrene inclusion, simulating a clinical application such as detection of breast microcalcifications. These phantom results and their corresponding Monte Carlo simulations underscore the utility of accessing both the fingerprint and high wavenumber regions for quantitative, depth-resolved biochemical analysis.

We previously published on the use of a Voigtian fitting scheme which employs a convolution of a Gaussian and a Lorentzian lineshape to approximate Raman spectral features and fit the five constituent water peaks as well as an N-H peak which falls within the same wavenumber region. In this exemplary embodiment, the Voigtian fitting is performed to the high wavenumber water band for phantoms with no upper layers and the maximum number of upper layers (5 upper layers˜2.5 mm thick) for phantoms that contain 50% and 75% water fraction. A greater contribution of deconvolved sub-bands associated with partially bound and free water relative to fully bound water is observed in phantoms with 75% water, and this effect is most pronounced when there is a greater depth of measurement (outer ring of collection fibers), or for shallower measurements when no upper layers are present. These preliminary experiments demonstrate the feasibility of using FP/HW SORS for depth-resolved assessment of water-tissue interactions.

Using optical phantoms, we have demonstrated that FP/HW SORS enables depth-resolved biochemical assessment including the molecular interactions of water. To our knowledge, this is the first implementation of FP/HW SORS in a probe-based configuration. While the exemplary study is for depth-resolved hydration and biochemical assessment and presents only initial validation of the system, it may enable numerous in vivo biomedical applications, such as diagnosing cancers which penetrate deeper in tissue with increasing disease progression and severity.

The foregoing description of the exemplary embodiments of the present invention has been presented only for the purposes of illustration and description and is not intended to be exhaustive or to limit the invention to the precise forms disclosed. Many modifications and variations are possible in light of the above teaching.

The embodiments are chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference is individually incorporated by reference.

### LIST OF REFERENCES

[1]. C. Choe, J. Lademann, and M. E. Darvin, “Depth profiles of hydrogen bound water molecule types and their relation to lipid and protein interaction in the human stratum corneum in vivo,” Analyst 141, 6329-6337 (2016).
[2]. L. E. Masson, C. M. O'Brien, I. J. Pence, J. L. Herington, J. Reese, T. G. van Leeuwen, and A. Mahadevan-Jansen, “Dual excitation wavelength system for combined fingerprint and high wavenumber Raman spectroscopy,” Analyst 143, 6049-6060 (2018).
[3]. P. Matousek, I. P. Clark, E. R. C. Draper, M. D. Morris, A. E. Goodship, N. Everall, M. Towrie, W. F. Finney, and A. W. Parker, “Subsurface Probing in Diffusely Scattering Media Using Spatially Offset Raman Spectroscopy,” Appl. Spectrosc. 59, 393-400 (2005).

What is claimed is:

1. A system for real-time assessment of systemic hydration, comprising:
a light source configured to operably emit light of first and second wavelengths;
means for delivering the emitted light to a target site to excite at least one first spot at the target site, and collecting Raman scattering light scattered from the target site at a plurality of second spots in response to excitation by the light;
a detector coupled with said means for obtaining a plurality of Raman spectra from the collected Raman scattering light, wherein each Raman spectrum is corresponding to a respective second spot of the target site, and associated with a depth of tissues at which the Raman scattering light is scattered; and
a controller coupled with the detector and configured to process the plurality of Raman spectra so as to identify spectral features from the plurality of Raman spectra, and assess systemic hydration from the identified spectral features.

2. The system of claim 1, wherein the light source comprise a dual wavelength laser module, wherein the first and second wavelengths are adapted such that when excited by the first wavelength light, the Raman scattering light corresponds to a fingerprint region; and when excited by the second wavelength light, the Raman scattering light corresponds to a high wavenumber region.

3. The system of claim 2, wherein the first wavelength is about 785 nm, and the second wavelength is in a range of about 660-700 nm.

4. The system of claim 2, wherein the dual wavelength laser module is configured to be operably switchable between the first wavelength and the second wavelength.

5. The system of claim 4, being capable of sequential or simultaneous acquisition of fingerprint and high wavenumber spectra, and collecting signal from multiple depths within the target site in a single acquisition.

6. The system of claim 1, wherein each second spot is apart from the at least one first spot so as to define a source-detection (S-D) offset distance between the at least one first spot excited with the light and the second spot from which the Raman scattering light is collected.

7. The system of claim 1, wherein the at least one first spot comprises a plurality of first spots.

8. The system of claim 1, wherein said means comprises an optical probe having a working end, coupled with the light source and configured to deliver the light to the target site to excite the at least one first spot proximal to the working end, and collect from the working end Raman scattering light scattered from the target site at one or more of the plurality of second spots in response to excitation.

9. The system of claim 8, wherein the optical probe comprises:
at least one source channel configured to deliver the light to the target site to excite the at least one first spot proximal to the working end; and
at least one collection channel configured to collect from the working end Raman scattering light scattered from the target site at the one or more of the plurality of second spots in response to excitation.

10. The system of claim 8, wherein the optical probe comprises:
at least one first fiber configured to deliver the light to the target site to excite the at least one first spot proximal to the working end; and
a plurality of second fibers configured to collect from the working end Raman scattering light scattered from the target site.

11. The system of claim 10, wherein the at least one first fiber and the plurality of second fibers are spatially arranged in a row, a matrix, a wing, or a ring form.

12. The system of claim 11, wherein the plurality of second fibers is spatially arranged in one or more collection rings surrounding the at least one first fiber.

13. The system of claim 12, wherein the plurality of second fibers is spatially arranged in three collection rings originated from the at least one first fiber with radii R1, R2 and R3 respectively.

14. The system of claim 13, wherein the optical probe is configured such that as the distance from the center of the probe increases, the number of collection fibers also increases to compensate for decreasing signal intensity with increasing offset.

15. The system of claim 13, wherein the inner, middle and outer rings are configured to measure hydration parameters from different depths.

16. The system of claim 10, wherein the fiber optic probe further comprises a shortpass or bandpass filter coupled to the at least one first fiber for delivering either the first wavelength light or the second wavelength light to the target site while preventing extraneous wavelengths of light from being transmitted.

17. The system of claim 16, wherein the shortpass or bandpass filter has a bandwidth of about 600-800 nm.

18. The system of claim 10, wherein the fiber optic probe further comprises a blocking filter coupled to the plurality of second fibers for preventing backscattered excitation light from being collected.

19. The system of claim 18, wherein the filter has an cut-on wavelength of about 800 nm.

20. The system of claim 1, wherein the detector comprises a spectrograph and/or a sensing member.

21. The system of claim 20, wherein the sensing member comprises at least one charge-coupled device (CCD), at least one complementary metal oxide semiconductor (CMOS), and/or at least one photodiode.

22. The system of claim 1, wherein the spectral features are associated with water, or electrolytes, or metabolic products, pus, or bacteria, or cells, and include spectral peaks in the high wavenumber region and the fingerprint region.

23. The system of claim 22, wherein the controller is configured to correlate, the identified peaks and spectral ratios for each depth of collection corresponding to the inner, outer and middle rings of the optical probe, so as to determine hydration based on these correlation values.

24. The system of claim 22, wherein the controller is configured to analyze the changes associated with hydration level using multivariate statistical, machine learning, deep learning or artificial intelligence (AI) approaches.

25. The system of claim 25, wherein the analysis utilizes generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, X is a matrix containing independent variables of BMI, age and temperature, p is a vector containing weight coefficients of the independent variables, and E is a residual error in the GLM, wherein a linear least squares regression is performed to choose p coefficients such that E is minimized.

26. The system of claim 22, wherein the controller is configured to perform Voigtian decomposition of the high wavenumber region of the Raman spectra into the spectral peaks including five component water peaks and an N-H peak in the same region, so as to analyze the distribution of fully-bound, partially bound, and free water molecules in the target site.

27. The system of claim 26, wherein the spectral features further includes spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

28. A method for real-time assessment of systemic hydration, comprising:
exciting a target site at at least one first spot with light of first and second wavelengths;
collecting Raman scattering light from the target site at a plurality of second spots, respectively, in response to illumination by the light;
obtaining the plurality of Raman spectra from the collected Raman scattering light, wherein each Raman spectrum is corresponding to a respective second spot of the target site, and associated with a depth of tissues at which the Raman light is scattered;
identifying spectral features from the plurality of Raman spectra; and
determining systemic hydration from the identified spectral features.

29. The method of claim 28, wherein the first and second wavelengths are adapted such that when excited by the first wavelength light, the Raman scattering light corresponds to a fingerprint region; and when excited by the second wavelength light, the Raman scattering light corresponds to a high wavenumber region.

30. The method of claim 29, wherein the first wavelength is about 785 nm, and the second wavelength is in a range of about 660-700 nm.

31. The method of claim 29, wherein the exciting step comprises exciting the target site at the at least one first spot with the first wavelength light and the second wavelength light sequentially, so that the fingerprint and high wavenumber spectra are sequentially or simultaneous acquired, and signals from multiple depths within the target site are collected in a single acquisition.

32. The method of claim 28, wherein the at least one first spot comprises a plurality of first spots.

33. The method of claim 28, wherein the exciting and collecting steps are performed with an optical probe having a working end.

34. The method of claim 33, wherein the optical probe comprises:
at least one source channel configured to deliver the light to the target site to excite the at least one first spot proximal to the working end; and
at one one collection channel configured to collect from the working end Raman scattering light scattered from the target site at one or more of the plurality of second spots in response to excitation.

35. The method of claim 33, wherein the optical probe comprises:
at least one first fiber configured to deliver the light to the target site to excite the at least one first spot proximal to the working end; and
a plurality of second fibers configured to collect from the working end Raman scattering light scattered from the target site.

36. The method of claim 35, wherein the at least one first fiber and the plurality of second fibers are spatially arranged in a row, a matrix, a wing, or a ring form.

37. The method of claim 35, wherein the plurality of second fibers is spatially arranged in one or more collection rings surrounding the at least one first fiber.

38. The method of claim 37, wherein the plurality of second fibers is spatially arranged in three collection rings originated from the at least one first fiber with radii R1, R2 and R3 respectively.

39. The method of claim 38, wherein the optical probe is configured such that as the distance from the center of the probe increases, the number of collection fibers also increases to compensate for decreasing signal intensity with increasing offset.

40. The method of claim 38, wherein the inner ring, middle and outer rings are configured to measure hydration parameters from different depths.

41. The method of claim 35, wherein the fiber optic probe further comprises a shortpass or bandpass filter coupled to the at least one first fiber for delivering either the first wavelength light or the second wavelength light to the target site while preventing extraneous wavelengths of light from being transmitted.

42. The method of claim 41, wherein the shortpass or bandpass filter has a bandwidth of about 600-800 nm.

43. The method of claim 35, wherein the fiber optic probe further comprises a blocking filter coupled to the plurality of second fibers for preventing backscattered excitation light from being collected.

44. The method of claim 43, wherein the filter has an cut-on wavelength of about 800 nm.

45. The method of claim 28, wherein the obtaining step is performed with a detector comprising a spectrograph and/or a sensing member.

46. The method of claim 45, wherein the sensing member comprises at least one charge-coupled device (CCD), at least one complementary metal oxide semiconductor (CMOS), and/or at least one photodiode.

47. The method of claim 28, wherein the spectral features are associated with water, or electrolytes, or metabolic products, pus, or bacteria, or cells, and include spectral peaks in the high wavenumber region and the fingerprint region.

48. The s method of claim 28, further comprising correlating the identified peaks and spectral ratios for each depth of collection corresponding to the inner, outer and middle rings of the optical probe, so as to determine t hydration based on these correlation values.

49. The method of claim 28, further comprising analyzing the changes associated with hydration level using multivariate statistical, machine learning, deep learning or artificial intelligence (AI) approaches.

50. The method of claim 49, wherein the analyzing step is performed with generalized linear models (GLM) that incorporates results from the Raman spectra and participant factors including body mass index (BMI), age and temperature, wherein the GLM is governed by a linear equation: Y=pX+E, wherein Y is a vector containing a dependent variable of the Raman spectra, X is a matrix containing independent variables of BMI, age and temperature, p is a vector containing weight coefficients of the independent variables, and E is a residual error in the GLM, wherein a linear least squares regression is performed to choose p coefficients such that E is minimized.

51. The method of claim 28, further comprising performing Voigtian decomposition of the high wavenumber region of the Raman spectra into the spectral peaks including five component water peaks and an N-H peak in the same region, so as to analyze the distribution of fully-bound, partially bound, and free water molecules in the target site.

52. The method of claim 51, wherein the spectral features further includes spectral ratios of an area under the curve (AUC) of the water peaks in the high wavenumber spectrum to the AUC of the entire high wavenumber spectrum, wherein the spectral ratios has a substantially linear relationship with respect to the percent water in the target site.

53. A device using Raman spectral features for real-time assessment of systemic hydration of a subject in biomedical applications, wherein the Raman spectral features are associated with water, electrolytes and metabolic products and include spectral peaks in the high wavenumber region and the fingerprint region, the device comprising:
a light source configured to emit light to excite a target site in the subject with an excitation wavelength that is determined based on wavenumber locations of the spectral peaks to be detected;
a filter configured, for each feature to be detected, to pass Raman scattering light of the wavenumber range at which that feature falls with respect to the given excitation wavelength, wherein the Raman scattering light is scattered from the target site in response to excitation by the light;
a detector configured to detect signals of the Raman scattering light passing through the filter; and
a controller configured to operate the light source and process the detected signals by the detector to determine systemic hydration of a subject in real time.

54. The device of claim 53, wherein the light source comprises a VCSEL-technology based single monochromatic light source.

55. The device of claim 53, further comprising dichroic mirrors (DMs) configured to direct the path of the emitted light.

56. The device of claim 53, further comprising an alert configured to generate an audible signal and/or a visible signal whenever the measured hydration level falls below what is determined to be normal for the subject.

57. The device of claim 53, wherein the detector comprises at least one charge-coupled device (CCD), at least one complementary metal oxide semiconductor (CMOS), and/or at least one photodiode.

58. The device of claim 53, wherein the filter comprises at least one linear variable filter (LVF), and/or at least one high-throughput narrow bandpass filter.

59. The device of claim 53, wherein the detector comprises a an avalanche photodiode (APD)., and/or a cooled linear diode array.

60. The device of claim 53, further comprising one or more slit apertures located between the filter and the detector.

61. The device of claim 53, being configured to accurately and noninvasively measure systemic hydration in a user and to send the measured results to one or more external devices for real-time remote monitoring, and/or data collection and storage.

62. The device of claim 53, being configured to be wearable and portable.
